(19) United States
(12) Patent Application Publication
(10) Pub. No.: US 2016/0206597 A1
(43) Pub. Date: Jul. 21, 2016

(54) New Use

(71) Applicants: Toni Lynne Bransford, Upper Montclair, NJ (US); Bryan BURKEY, Winchester, MA (US); Thomas LANGENICKEL, Bottmingen (CH); Martin LEFKOWITZ, Suffern, NY (US); Victor Chengwei SHI, Mendham, NJ (US)

(72) Inventors: Toni Lynne Bransford, Upper Montclair, NJ (US); Bryan BURKEY, Winchester, MA (US); Thomas LANGENICKEL, Bottmingen (CH); Martin LEFKOWITZ, Suffern, NY (US); Victor Chengwei SHI, Mendham, NJ (US)

(21) Appl. No.: 14/914,029

(22) PCT Filed: Aug. 26, 2013
(86) PCT No.: PCT/US2013/056680

§ 371 Date: Feb. 24, 2016

(51) Int. Cl. A61K 31/41; A61K 31/216

(52) U.S. Cl. CPC A61K 31/41; A61K 31/216

(57) ABSTRACT

The present invention relates to methods and pharmaceutical compositions for renal protection in a mammal in need thereof, such as a mammal having a disease manifested by atrial enlargement and/or remodeling or suffering from hypertension or heart failure or being prone to suffering from hypertension and/or heart failure, comprising administration of a therapeutically effective amount, or a prophylactically effective amount, of an Angiotensin Receptor Neprilysin inhibitor (ARNi) or of a combination of an Angiotensin Receptor Blocker (ARB) with a Neutral Endopeptidase inhibitor (NEPi) or with a NEPi pro-drug to said mammal.

The present invention relates to methods and pharmaceutical compositions for renal protection in a mammal in need thereof, such as a mammal having a disease manifested by atrial enlargement and/or remodeling or suffering from hypertension or heart failure or being prone to suffering from hypertension and/or heart failure, comprising administration of a therapeutically effective amount, or a prophylactically effective amount, of an Angiotensin Receptor Neprilysin inhibitor (ARNi) or of a combination of an Angiotensin Receptor Blocker (ARB) with a Neutral Endopeptidase inhibitor (NEPi) or with a NEPi pro-drug to said mammal.

### BACKGROUND OF THE INVENTION

As the population lives longer resulting in an increased prevalence of cardiovascular risk factors and disease, and as survival following acute myocardial infarction (MI) increases, the numbers of patients living with congestive heart failure (CHF) is expanding. For example, risk factors, such as hypertension, are common prognostic comorbidities in chronic HF. In parallel, a concomitant increase in the number of hospitalizations for acute decompensated heart failure (ADHF) has occurred. In the United States alone, heart failure (HF) affects 5.7 million Americans, with over 650,000 new cases diagnosed annually, with increasing hospitalization rates.

Heart failure remains a high unmet medical need with an annual mortality rate of about 20%. Reductions in mortality and cardiovascular morbidity have been achieved by RAAS blockers (Angiotensin Converting Enzyme (ACE) inhibitors and Angiotensin Receptor Blockers (ARBs)) and beta (β)-blockers in HF. While survival rates have improved for HF with reduced ejection fraction (HF-REF) over recent decades, due to more widespread use of drugs that block the renin-angiotensin-aldosterone system (RAAS) and improved acute care, residual mortality rates remain high. For patients with HF with preserved ejection fraction (HF-PEF) no therapy has proven to be effective at reducing morbidity and mortality. Overall, the therapeutic benefits of RAAS blockade with ACE inhibitors and/or ARBs remain limited, possibly caused by (a) angiotensin II escape due to incomplete ACE inhibition or angiotensin II originating from alternative non-ACE pathways, and (b) other neuro-hormonal and other mechanisms contributing to cardiac disease and outcomes.

The consequences of activation of the RAAS and sympathetic nervous system in the pathogenesis of HF are well established, as is the therapeutic benefit of RAAS blockers in improving HF outcomes: Chronic HF is a progressive condition characterized by elevated cardiac filling pressures, and reduced cardiac output and tissue oxygen delivery [Schrier, R. W. et al. (2000) Therapy of heart failure. Kidney Int. 57, 1418-1425]. These hemodynamic abnormalities result in activation of the RAAS and sympathetic nervous systems to maintain vital organ perfusion. Initially, this serves as an acute compensatory response, but prolonged activation contributes to the pathobiology of HF, resulting in progressive cardio-renal abnormalities, including myocardial hypertrophy, fibrosis and apoptosis, increased systemic vascular resistance, and increased sodium and water retention [Brewster, U. C. et al. (2003) The renin-angiotensin-aldosterone system: cardio-renal effects and implications for renal and cardiovascular disease states. Am. J. Med. Sci. 326, 15-24].

On the other hand, there is growing evidence of the involvement of the Natriuretic Peptide (NP) system in the pathogenesis of Heart Failure: The NP system consists primarily of three well-characterized peptides, with each being a distinct gene product with structural similarity: atrial natriuretic peptide (ANP) and B-type natriuretic peptide (BNP) are mainly from cardiomyocytes, and C-type natriuretic peptide (CNP) is mostly from endothelial and renal cells. As filling pressures rise in HF, increased cardiac stretch causes the secretion of precursor NPs, which are cleaved by specific proteases to produce biologically active NPs which then act on NP receptors (NP receptor-A [NPR-A], NPR-B and NPR-C). Binding of NPs to NPR-A and NPR-B activates particulate guanylate cyclase resulting in increases in the second messenger, cyclic guanosine monophosphate (cGMP), which mediates many of the cardiovascular and renal effects of the NPs. NPs are cleared from the circulation by two mechanisms binding to NPR-C and inactivation (hydrolytic cleavage) by neprilysin. Neprilysin has a high affinity for both ANP and CNP, and a lower affinity for BNP, which is more resistant to hydrolysis. Since N-terminal pro-BNP (NT-proBNP) is not a substrate of neprilysin, it can be a useful as a cardiac biomarker to assess therapeutic effect and prognosis in patients treated with neprilysin inhibitors.

The cardiovascular and renal effects of the NP system oppose those of the RAAS [Nathisuwan, S. and Talbert, R. L. (2002) A review of vasopeptidase inhibitors: a new modality in the treatment of hypertension and chronic heart failure. Pharmacotherapy 22, 27-42], providing the scientific and therapeutic basis for neprilysin inhibition in the setting of HF. One of the major effects of NPs is vasodilation, which results from cGMP-mediated relaxation of smooth muscle cells as well as indirect effects of NPs to inhibit the RAAS and decrease endothelin-1 (ET-1) production. Indeed, NPs have been shown to cause significant reductions in systemic vascular resistance, pulmonary artery pressure, pulmonary capillary wedge pressure and right arterial pressure in patients with severe HF. NPs have also been shown to mediate other beneficial hemodynamic effects, including reducing arterial stiffness and enhancing endothelial function [Rubattu, S. et al. (2008) Natriuretic peptides: an update on bioactivity, potential therapeutic use, and implication in cardiovascular diseases. Am. J. Hypertens. 21, 733-741].

NPs promote sodium and water excretion by inhibiting sodium reabsorption in the proximal and distal nephron, while preventing decreases in glomerular filtration rate by regulating tubuloglomerular feedback. These effects of NPs have been observed in patients with severe HF, resulting in improvement in hemodynamics and renal function. In addition to the direct effects of NPs on the kidney, their inhibitory actions on the RAAS and sympathetic nervous system also contribute to their natriuretic, diuretic and hemodynamic effects.

Generally it was thought that the NP system was up-regulated in HF due to high circulating levels of total immune-reactive ANP and BNP, but more recent studies indicate that mature BNP (BNP1-32) (or biologically active BNP) levels are reduced and levels of less biologically-active BNP fragments are increased. Thus, advanced HF may represent a state of NP deficiency. Furthermore, the expression and activation of neprilysin are increased in patients with HF, which enhances the rate of degradation of NPs and contributes to reduced levels of biologically active NPs [Mangiafico, S. et al. (2013) Neutral endopeptidase inhibition and the natriuretic peptide system: an evolving strategy in cardiovascular therapeutics. Eur. Heart J. 34, 886-893].

As HF progresses, relative resistance or hypo-responsiveness to NPs develops, which is particularly evident in the kidney and vasculature. This hypo-responsiveness is an important feature of HF that adversely affects prognosis by worsening sodium retention and volume overload and increasing peripheral vascular resistance. The mechanisms for NP resistance are multifactorial and include: down-regulation of NP receptors, dysregulated NP signal transduction, increased cGMP degradation and activation of the RAAS.

In addition to hydrolyzing the NPs, neprilysin also hydrolyzes other vasoactive peptides, including substance P, bradykinin, ET-1, angiotensin I (Ang I) and Ang II. Since there are multiple neprilysin substrates with differing and, in some instances, opposing biologic actions, the pharmacologic profile of neprilysin inhibitors is complex and will depend on the net effect on all biologically relevant substrates: While inhibition of neprilysin is expected to result in beneficial cardiovascular and renal effects in HF by increasing NP levels, corresponding increases in Ang II and ET-1, both of which have vasoconstrictor, pro-fibrotic and pro-hypertrophic properties, would be expected to oppose the beneficial effects of the NPs. In the case of angiotensin, neprilysin hydrolyzes and inactivates Ang II; therefore, neprilysin inhibition alone will not only increase NP levels but can also result in accumulation of Ang II, which could attenuate or negate any beneficial NP effects in the setting of HF. It should also be noted that both substance P and bradykinin, which are both inactivated by neprilysin, have vasodilatory properties, increase vascular permeability and, when combined with an angiotensin-converting-enzyme inhibitor (ACEI), are implicated in the pathogenesis of angioedema, a potential side effect of neprilysin inhibitors.

These findings have led to the development of a novel class of drugs that combines the actions of NEP inhibitors and ARBs, known as angiotensin receptor blockade with neutral endopeptidase inhibition (ARNi) foreseen for the treatment of heart failure and hypertension. These innovative agents are aimed to better control blood pressure (BP) and also have a therapeutic potential in the setting of HF without increasing the risk of angioedema, which is commonly seen in case of concomitant inhibition of neprilysin and of ACE as mentioned above.

The compounds and pharmaceutical compositions disclosed herein include novel drug candidates useful for the treatment of hypertension and/or heart failure. Such compounds or pharmaceutical compositions have been previously disclosed in WO 2003/059345, WO 2007/056546, and WO 2009/061713, which are herein incorporated by reference.

However, in order to enhance current therapies for cardiovascular diseases, such agents should also confer renal protection whilst providing optimal cardiovascular benefits: Decreased renal function has consistently been found to be an independent risk factor for cardiovascular (CV) disease outcomes and all-cause mortality in a large spectrum of cardiovascular patients including patients with HF (Anavekar et al 2004, Go et al 2004). Furthermore, renal functional impairment in patients with hypertension or HF often limits the use of treatments known to improve blood pressure control or the outcomes in HF. Therefore, a new therapy that not only provides the benefit of reducing CV mortality and morbidity, but also provides an additional renal-protective effect would be considered highly valuable for HF patients. It would fulfill an important unmet medical need in this population.

Accordingly, there is still a need for new therapies and pharmaceutical compositions providing renal protection in patients suffering from cardiovascular diseases such as heart failure and hypertension, while concomitantly being useful for the treatment of said diseases.

### SUMMARY OF THE INVENTION

Surprisingly, the administration of a pharmaceutical composition comprising a therapeutically effective amount, or a prophylactically effective amount, of an Angiotensin Receptor Neprilysin inhibitor (ARNi) as defined herein or of a therapeutically effective amount, or a prophylactically effective amount, of a combination of an Angiotensin Receptor Blocker (ARB) with a Neutral Endopeptidase inhibitor (NEPi) or with a NEPi pro-drug, in a 1:1 molar ratio, as defined herein, to patients in need thereof has shown to provide renal protection. Renal protection was shown by inducing a lesser decrease of the estimated glomerular filtration rate (eGFR) than administration of the corresponding amount of the angiotensin receptor blocker alone. In addition, the administration also proved to reduce the left atrial volume, the left atrial volume index (LAVI) and the left atrial dimension in patients with heart failure with preserved ejection fraction (HF-PEF).

Said pharmaceutical composition comprises

a) a therapeutically effective amount of the compound of the formula (I)

[(A₁)(A₂)](Na)_{y}.xH₂O (I)
wherein
A₁ is S—N-valeryl-N-{[2′-(1H-tetrazole-5-yl)-biphenyl-4-yl]-methyl}-valine in the anion form;
A₂ is (2R,4S)-5-biphenyl-4-yl-4-(3-carboxy-propionylamino)-2-methyl-pentanoic acid ethyl ester in the anion form;
Na is a sodium ion;
y is 1 to 3; and
x is 0 to 3;
   or
   b) a combination comprising a therapeutically effective amount of a 1:1 molar ratio
(i) of valsartan or a pharmaceutically acceptable salt thereof; and
(ii) of N-(3-carboxy-1-oxopropyl)-(45)-(p-phenylphenylmethyl)-4-amino-2R-methylbutanoic acid ethyl ester or (2R,4S)-5-biphenyl-4-yl-4-(3-carboxy-propionyl amino)-2-methyl-pentanoic acid or a pharmaceutically acceptable salt thereof.

In one embodiment, the mammal is a human.

In another embodiment, the compound of formula (I) is trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate (LCZ696).

In one embodiment, the mammal has a disease characterized and/or manifested by atrial enlargement and/or remodeling.

In another embodiment, the present invention is directed to the pharmaceutical composition as defined above for the renal protection of a mammal in need thereof, preferably a mammal having a disease manifested by atrial enlargement and/or remodeling.

In another embodiment, the present invention is directed to the use of the pharmaceutical composition as defined above for the manufacture of a medicament for the renal protection of a mammal in need thereof, preferably a mammal having a disease manifested by atrial enlargement and/or remodeling.

In another embodiment, the present invention is directed to a pharmaceutical composition comprising:

a) a therapeutically effective amount of the compound of the formula

[(A₁)(A₂)](Na)_{y}.xH₂O (I)
wherein
A₁ is S—N-valeryl-N-{[2′-(1H-tetrazole-5-yl)-biphenyl-4-yl]-methyl}-valine in the anion form;
A₂ is (2R,4S)-5-biphenyl-4-yl-4-(3-carboxy-propionylamino)-2-methyl-pentanoic acid ethyl ester in the anion form;
Na is a sodium ion;
y is 1 to 3; and
x is 0 to 3;
   or
   b) a combination of
(i) a therapeutically effective amount valsartan or a pharmaceutically acceptable salt thereof; and
(ii) a therapeutically effective amount of N-(3-carboxy-1-oxopropyl)-(4S)-(p-phenylphenylmethyl)-4-amino-2R-methylbutanoic acid ethyl ester or (2R,4S)-5-biphenyl-4-yl-4-(3-carboxy-propionyl amino)-2-methyl-pentanoic acid or a pharmaceutically acceptable salt thereof,
   for the protection of the kidney in a mammal.

In one embodiment, the pharmaceutical composition comprises in addition one or more pharmaceutically acceptable carriers.

In one embodiment, the pharmaceutical composition comprises the compound of formula (I) which is trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate (LCZ696).

### DETAILED DESCRIPTION OF THE INVENTION

### Methods of Treatment

The present invention is based upon the surprising and unexpected finding that certain drugs (i.e. LCZ696) effective for the treatment of cardiovascular disease or conditions, such as heart failure or hypertension, in human subjects in addition provide renal protection thereby enhancing the treatment potential for cardiovascular diseases. Renal protection was shown by inducing a lesser decrease of the estimated glomerular filtration rate (eGFR) than administration of the corresponding amount of the angiotensin receptor blocker alone. In addition, the administration also proved to reduce the left atrial volume, the left atrial volume index (LAVI) and the left atrial dimension in patients with heart failure with preserved ejection fraction (HF-PEF).

Thus, the invention encompasses a method for protection of the kidney in a mammal comprising administering to said mammal in need of such protection a pharmaceutical composition comprising a therapeutically effective amount, or a prophylactically effective amount, of an Angiotensin Receptor Neprilysin inhibitor (ARNi) as defined herein or of a therapeutically effective amount, or a prophylactically effective amount, of a combination of an Angiotensin Receptor Blocker (ARB) with a Neutral Endopeptidase inhibitor (NEPi) or with a NEPi pro-drug, in a 1:1 molar ratio, as defined herein, to patients in need thereof.

Said pharmaceutical composition comprises

a) a therapeutically effective amount of the compound of the formula (I)

[(A₁)(A₂)](Na)_{y}.xH₂O (I)
wherein
A₁ is S—N-valeryl-N-{[2′-(1H-tetrazole-5-yl)-biphenyl-4-yl]-methyl}-valine in the anion form;
A₂ is (2R,4S)-5-biphenyl-4-yl-4-(3-carboxy-propionylamino)-2-methyl-pentanoic acid ethyl ester in the anion form;
Na is a sodium ion;
y is 1 to 3; and
x is 0 to 3;
   or
   b) a combination comprising a therapeutically effective amount of a 1:1 molar ratio
(i) of valsartan or a pharmaceutically acceptable salt thereof; and
(ii) of N-(3-carboxy-1-oxopropyl)-(4S)-(p-phenylphenylmethyl)-4-amino-2R-methylbutanoic acid ethyl ester or (2R,4S)-5-biphenyl-4-yl-4-(3-carboxy-propionyl amino)-2-methyl-pentanoic acid or a pharmaceutically acceptable salt thereof.

In another embodiment, the compound of formula (I) is trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate (LCZ696).

In one embodiment, the mammal is a human.

In one embodiment, the mammal has a disease characterized and/or manifested by atrial enlargement and/or remodeling.

Diseases characterized by atrial enlargement and/or remodeling include, but are not limited to heart failure, cardiac dysrhythmias; mitral stenosis and regurgitation, cardiomyopathies, hypertension and pulmonary heart diseases. In one embodiment, cardiac dysrhythmias comprise atrial fibrillation, new onset atrial fibrillation and recurrent atrial fibrillation. In one embodiment, heart failure comprises congestive heart failure, left heart failure, right heart failure, chronic heart failure, advanced heart failure, acute heart failure, acute decompensated heart failure, heart failure with reduced ejection fraction (HF-REF), and heart failure with preserved ejection fraction (HF-PEF). In particular, heart failure comprises heart failure with preserved ejection fraction (HF-PEF) and heart failure with reduced ejection fraction (HF-REF).

In one embodiment, the mammal suffers from hypertension or heart failure or is prone to suffering from hypertension and/or heart failure. In one embodiment said patients suffering from heart failure are patients suffering from heart failure with preserved ejection fraction (HF-PEF) or heart failure with reduced ejection fraction (HF-REF). In one embodiment said patients suffering from heart failure are patients suffering from heart failure with preserved ejection fraction (HF-PEF).

In another embodiment, the mammal suffers from hypertension.

In another embodiment, the mammal has an enlarged heart.

In another embodiment, the mammal has atherosclerosis.

The present invention provides that the therapeutically effective amount of compound of formula (I) or of the combination is effective to provide renal protection to the mammal.

The present invention also provides that the therapeutically effective amount of compound of formula (I) or of the combination is effective to induce at least one physiological effect in the mammal including vasodilation, diuresis, natriuresis and combinations thereof.

The present invention also provides that the therapeutically effective amount of compound of formula (I) is effective to inhibit one or more physiological mechanisms in the human subject including vasoconstriction, remodulation, hypertrophy, hyperproliferation, edema, and combinations thereof.

The present invention also provides that the therapeutically effective amount of compound of formula (I) or of the combination better preserves renal function than the corresponding amount of an angiotensin receptor blocker, such as valsartan, alone.

In one embodiment, the administration of a therapeutically effective amount of compound of formula (I) or of the combination slows the time to a change in renal function defined by loss of estimated glomerular filtration rate (eGFR). Such slowdown in change of renal function is preferably in addition to reducing CV mortality and HF hospitalization. Such behavior can be seen for example in comparison to the administration of the corresponding amount of an angiotensin receptor blocker, such as valsartan, alone.

In one embodiment, the administration of a therapeutically effective amount of compound of formula (I) or of the combination induces a lesser decrease of the estimated glomerular filtration rate (eGFR) than administration of the corresponding amount of an angiotensin receptor blocker, such as valsartan, alone.

In another embodiment, the administration of a therapeutically effective amount of compound of formula (I) or of the combination slows down decrease of the estimated glomerular filtration rate (eGFR) in patients suffering from heart failure, preferably from heart failure with preserved ejection fraction (HF-PEF). Such slowdown in change of renal function is preferably in addition to reducing CV mortality and HF hospitalization. Such behavior can be seen for example in comparison to the administration of the corresponding amount of an angiotensin receptor blocker, such as valsartan, alone.

Accordingly, in another embodiment, the administration of a therapeutically effective amount of compound of formula (I) or of the combination induces a slower decrease of the estimated glomerular filtration rate (eGFR) in patients suffering from heart failure, preferably from heart failure with preserved ejection fraction (HF-PEF), than administration of the corresponding amount of an angiotensin receptor blocker, such as valsartan, alone.

In a further embodiment, the administration of a therapeutically effective amount of compound of formula (I) or of the combination is—in addition to the renal protection—effective to achieve a reduction of the left atrial volume, the left atrial volume index (LAVI) and/or of the left atrial dimension. In one embodiment such reduction is larger than upon administration of the corresponding amount of an angiotensin receptor blocker, such as valsartan, alone.

In a further embodiment, the administration of the therapeutically effective amount of compound of formula (I) or of the combination is effective to simultaneously provide renal protection and cardiovascular benefit to the mammal.

In another embodiment, the administration of a therapeutically effective amount of compound of formula (I) or of the combination is—in addition to the renal protection—effective to achieve a reduction of systolic blood pressure (SDP) and/or diastolic blood pressure (DBP). In one embodiment, such reduction is larger than upon administration of the corresponding amount of an angiotensin receptor blocker, such as valsartan, alone.

In one aspect of the aforementioned embodiments, the mammal or patient has a disease characterized and/or manifested by atrial enlargement and/or remodelling. Diseases characterized by atrial enlargement and/or remodelling include, but are not limited to heart failure, cardiac dysrhythmias; mitral stenosis and regurgitation, cardiomyopathies, hypertension and pulmonary heart diseases. In one embodiment, cardiac dysrhythmias comprise atrial fibrillation, new onset atrial fibrillation and recurrent atrial fibrillation. In one embodiment, heart failure comprises congestive heart failure, left heart failure, right heart failure, chronic heart failure, advanced heart failure, acute heart failure, acute decompensated heart failure, heart failure with reduced ejection fraction (HF-REF), and heart failure with preserved ejection fraction (HF-PEF). In particular, heart failure comprises heart failure with preserved ejection fraction (HF-PEF) and heart failure with reduced ejection fraction (HF-REF). In one embodiment thereof, the mammal suffers from hypertension or heart failure or is prone to suffering from hypertension and/or heart failure. In one embodiment said patients suffering from heart failure are patients suffering from heart failure with preserved ejection fraction (HF-PEF) or heart failure with reduced ejection fraction (HF-REF). In one embodiment said patients suffering from heart failure are patients suffering from heart failure with preserved ejection fraction (HF-PEF). In another embodiment thereof, the mammal suffers from hypertension.

According to one aspect of the present invention, the administration of a therapeutically effective amount of compound of formula (I) or of the combination is superior to valsartan alone and/or enalapril alone in achieving renal protection in addition to reducing blood pressure, reducing CV mortality and/or reducing HF hospitalization.

According to another aspect of the present invention, the administration of a therapeutically effective amount of compound of formula (I) or of the combination provides renal protection and when administered or used in the context of the invention leads to a sustained reduction in plasma NT-proBNP concentration.

In a further embodiment of the present invention the pharmaceutical composition comprises in addition one or more pharmaceutically acceptable carriers.

In a further embodiment of the present invention the pharmaceutical composition is administered in a form to deliver a daily overall dose of LCZ696 from 50 mg to 400 mg. In one embodiment thereof, the pharmaceutical composition is administered to deliver LCZ696 twice daily with a dose of 50 mg, 100 mg, or 200 mg.

### DEFINITIONS

Throughout this specification and in the claims that follow, the following terms are defined with the following meanings, unless explicitly stated otherwise.

The term “prevention” refers to prophylactic administration to a healthy subject to prevent the development of the conditions mentioned herein. Moreover, the term “prevention” means prophylactic administration to patients being in a pre-stage of the conditions to be treated.

The term “delay of progression”, as used herein, refers to administration to patients being in a pre-stage of the condition to be treated in which patients with a pre-form of the corresponding condition is diagnosed.

The term “treatment” is understood the management and care of a patient for the purpose of combating the disease, condition or disorder.

The term “therapeutically effective amount” refers to an amount of a drug or a therapeutic agent that will elicit the desired biological and/or medical response of a tissue, system or an animal (including man) that is being sought by a researcher or clinician.

The terms “mammal” include, but are not limited to, humans, dogs, cats, horses, pigs, cows, monkeys, rabbits and mice. The preferred mammals are humans.

The terms “administration of” and or “administering a” compound should be understood to mean providing a compound of the invention or a pharmaceutically acceptable salt or ester thereof, or a pro-drug thereof to a subject in need of treatment. The administration of the composition of the present invention in order to practice the present methods of therapy is carried out by administering a therapeutically effective amount of the compounds in the composition to a subject in need of such treatment or prophylaxis. The need for a prophylactic administration according to the methods of the present invention is determined via the use of well-known risk factors. The effective amount of an individual compound is determined, in the final analysis, by the physician in charge of the case, but depends on factors such as the exact disease to be treated, the severity of the disease and other diseases or conditions from which the patient suffers, the chosen route of administration, other drugs and treatments which the patient may concomitantly require, and other factors in the physician's judgment.

The term “prophylactically effective amount” as used herein means the amount of the active compounds in the composition that will elicit the biological or medical response in a tissue, system, subject, or human that is being sought by the researcher, veterinarian, medical doctor or other clinician, to prevent the onset of a disease characterized and/or manifested by atrial enlargement and/or remodeling.

The term “pharmaceutically acceptable”, as used herein, refers to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk ratio.

The term “eGFR” refers to estimated glomerular filtration rate. Within the context of the present invention the eGFR is calculated by the Modification in Diet in Renal Disease (MDRD) formula, which is the one recommended by NICE and The Renal Association (UK), and which is based on the equation described in Levey A S, Bosch J P, Lewis J B, et al; “A more accurate method to estimate glomerular filtration rate from serum creatinine: a new prediction equation. Modification of Diet in Renal Disease Study Group.” Ann Intern Med. 1999 Mar. 16; 130(6):461-70.

The term “Rate of change in eGFR” or “eGFR slope” refers to the rate of change (slope) in eGFR from baseline to endpoint during a certain study period. For the rate change in eGFR, the eGFR slope will be calculated by fitting the patient's eGFR assessments with a linear regression model with time as the independent variable. The derived eGFR slopes will be then analyzed using an ANCOVA model with treatment and region as fixed effect factors, and baseline eGFR value as a covariate. The estimated treatment effects, based on the least-squared means for within and between treatment groups, and the corresponding two-sided 95% confidence intervals will be provided.

The New York Heart Association (NYHA) classification grades the severity of heart failure symptoms as one of four functional classes. The NYHA classification is widely used in clinical practice and in research because it provides a standard description of severity that can be used to assess response to treatment and to guide management. The New York Heart Association functional classification based on severity of symptoms and physical activity:

Class I: No limitation of physical activity. Ordinary physical activity does not cause undue breathlessness, fatigue, or palpitations.
Class II: Slight limitation of physical activity. Comfortable at rest, but ordinary physical activity results in undue breathlessness, fatigue, or palpitations.
Class III: Marked limitation of physical activity. Comfortable at rest, but less than ordinary physical activity results in undue breathlessness, fatigue, or palpitations.
Class IV: Unable to carry on any physical activity without discomfort. Symptoms at rest can be present. If any physical activity is undertaken, discomfort is increased.

### Compounds and Compositions for Use According to the Invention

The compounds of the invention used in the aforementioned methods are

a) a compound of the formula (I)

[(A₁)(A₂)](Na)_{y}.xH₂O (I)
wherein
A₁ is S—N-valeryl-N-{[2′-(1H-tetrazole-5-yl)-biphenyl-4-yl]-methyl}-valine in the anion form;
A₂ is (2R,4S)-5-biphenyl-4-yl-4-(3-carboxy-propionylamino)-2-methyl-pentanoic acid ethyl ester in the anion form;
Na is a sodium ion;
y is 1 to 3, preferably 1, 2, or 3; and
x is 0 to 3, preferably 0, 0.5, 1, 1.5, 2, 2.5, or 3;
   or
   b) a combination comprising a 1:1 molar ratio
(i) of valsartan or a pharmaceutically acceptable salt thereof; and
(ii) of N-(3-carboxy-1-oxopropyl)-(4S)-(p-phenylphenylmethyl)-4-amino-2R-methylbutanoic acid ethyl ester or (2R,4S)-5-biphenyl-4-yl-4-(3-carboxy-propionyl amino)-2-methyl-pentanoic acid or a pharmaceutically acceptable salt thereof.

In one embodiment, y is 3 and x is 2.5.

In another embodiment, the compound of formula (I) is trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate (LCZ696).

In another embodiment, the combination comprises a 1:1 molar ratio
(i) of valsartan; and
(ii) of N-(3-carboxy-1-oxopropyl)-(4S)-(p-phenylphenylmethyl)-4-amino-2R-methylbutanoic acid ethyl ester or a pharmaceutically acceptable salt thereof, such as sodium or calcium salt.

In a preferred embodiment, the invention encompasses a pharmaceutical composition for use in protecting the kidney in a mammal or human subject as described herein, the composition comprising a therapeutically effective amount of trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate (Compound LCZ696). Such compounds and pharmaceutical compositions have been previously disclosed in WO2007/056546 and WO 2009/061713, whose preparative teachings are incorporated herein by reference.

In a further embodiment of the invention, the pharmaceutical compositions for use according to the present invention comprise trisodium [3-((1S,3R)-1-biphenyl-4-ylmethyl-3-ethoxycarbonyl-1-butylcarbamoyl) propionate-(S)-3′-methyl-2′-(pentanoyl{2″-(tetrazol-5-ylate)biphenyl-4′-ylmethyl}amino)butyrate] hemipentahydrate (LCZ696) and deliver upon administration the NEP inhibitor pro-drug and the angiotensin receptor blocker together to the patient.

The pharmaceutical compositions for use according to the present invention comprise a therapeutically effective amount of a compound of the formula (I) or of a combination of valsartan and the NEP inhibitor or NEP inhibitor as defined herein above or a pharmaceutically acceptable salt or ester thereof, or pro-drug thereof. Each dosage unit can contain the daily dose or may contain a fraction of the daily dose, such as one-half or one-third of the dose.

In one embodiment of the invention for all of its uses, the pharmaceutical composition comprises the the NEP inhibitor pro-drug N-(3-carboxy-1-oxopropyl)-(4S)-p-phenylphenylmethyl)-4-amino-(2R)-methylbutanoic acid ethyl ester or the NEP inhibitor N-(3-carboxy-1-oxopropyl)-(4S)-p-phenylphenylmethyl)-4-amino-(2R)-methylbutanoic acid, or pharmaceutically acceptable salts thereof, and the Angiotensin Receptor Blocker valsartan or a pharmaceutically acceptable salt thereof. Such combinations are for example disclosed within international patent application WO 2003/059345, which is herewith incorporated by reference.

In one embodiment, the pharmaceutical composition comprises the NEP inhibitor pro-drug N-(3-carboxy-1-oxopropyl)-(4S)-p-phenylphenylmethyl)-4-amino-(2R)-methylbutanoic acid ethyl ester or the NEP inhibitor N-(3-carboxy-1-oxopropyl)-(4S)-p-phenylphenylmethyl)-4-amino-(2R)-methylbutanoic acid, or pharmaceutically acceptable salts thereof, and the Angiotensin Receptor Blocker valsartan or a pharmaceutically acceptable salt thereof, in a 1:1 molar ratio.

(i) Valsartan or (S)—N-valeryl-N-{[2′-(1H-tetrazole-5-yl)-biphenyl-4-yl]-methyl}-valine) or a pharmaceutically acceptable salt thereof that can be purchased from commercial sources or can be prepared according to known methods, such as described in U.S. Pat. No. 5,399,578 and EP 0443983, whose preparative teachings are incorporated by reference herein. Valsartan may be used in certain embodiments of the invention in its free acid form, as well as in any suitable salt form. Depending upon the circumstance, esters or other derivatives of the carboxylic grouping may be employed as well as salts and derivatives of the tetrazole grouping.
(ii) N-(3-carboxy-1-oxopropyl)-(4S)-(p-phenylphenylmethyl)-4-amino-2R-methylbutanoic acid ethyl ester or (2R,4S)-5-biphenyl-4-yl-4(3-carboxy-propionyl amino)-2-methyl-pentanoic acid can be prepared by known methods such as described in U.S. Pat. No. 5,217,996 which is herein incorporated by reference. Either compound may be admixed with valsartan to prepare compounds of the formula (i)/(ii). Compounds 5-biphenyl-4-yl-4(3-carboxy-propionyl amino)-2-methyl-pentanoic acid can exist as the (2R,4S), (2R,4S), (2R,4S) or (2R,4S) isomer. Preferred is N-(3-carboxy-1-oxopropyl)-(4S)-(p-phenylphenylmethyl)-4-amino-2R-methylbutanoic acid ethyl ester. These compounds may be used for purposes of this invention in its free or ester form. The corresponding active ingredient or a pharmaceutically acceptable salt thereof may also be used in the form of a hydrate or include other solvents used for crystallization.

Preferably, compound (I) or LCZ696, or compounds (i)/(ii) are substantially pure or in a substantially pure form. As used herein, “substantially pure” refers to at least about 90% purity, more preferably at least about 95% and most preferably at least about 98% purity.

Also preferred is that compound (I) or L, or compounds (i)/(ii) are solid or a solid form or solid state. The solid, solid form or solid state can be crystalline, partially crystalline, amorphous or poly-amorphous, preferably in the crystalline form.

The pharmaceutical compositions according to the invention can be prepared in a manner known per se and are those suitable for enteral, such as oral or rectal, and parenteral administration to mammals (warm-blooded animals), including man, comprising a therapeutically effective amount of the pharmacologically active compound, alone or in combination with one or more pharmaceutically acceptable carriers, especially suitable for enteral or parenteral application.

The pharmaceutical preparations of the invention contain, for example, from about 0.1% to about 100%, e. g. 80% or 90%, or from about 1% to about 60%, of the active ingredient. The term “about” or “approximately”, as used herein in each instance, shall have the meaning of within 10%, more preferably within 5%, of a given value or range.

Pharmaceutical preparations according to the invention for enteral or parenteral administration are, e.g., those in unit dose forms, such as sugar-coated tablets, tablets, capsules, bars, sachets, granules, syrups, aqueous or oily suspensions or suppositories and furthermore ampoules. These are prepared in a manner known per se, e. g. by means of conventional mixing, granulating, sugar-coating, dissolving or lyophilizing processes. Thus, pharmaceutical preparations for oral use can be obtained by combining the active ingredient with solid carriers, if desired granulating a mixture obtained, and processing the mixture or granules, if desired or necessary, after addition of suitable excipients to give tablets or sugar-coated tablet cores.

Tablets may be formed from the active compound with fillers, for example calcium phosphate; disintegrating agents, for example maize starch, lubricating agents, for example magnesium stearate; binders, for example microcrystalline cellulose or polyvinylpyrrolidone and other optional ingredients known in the art to permit tabletting the mixture by known methods. Similarly, capsules, for example hard or soft gelatin capsules, containing the active compound with or without added excipients, may be prepared by known methods. The contents of the capsule may be formulated using known methods so as to give sustained release of the active compound.

Other dosage forms for oral administration include, for example, aqueous suspensions containing the active compound in an aqueous medium in the presence of a non-toxic suspending agent such as sodium carboxymethylcellulose, and oily suspensions containing the active compounds in a suitable vegetable oil, for example arachis oil.

The active compound may be formulated into granules with or without additional excipients. The granules may be ingested directly by the patient or they may be added to a suitable liquid carrier (e.g. water) before ingestion. The granules may contain disintegrants, e.g. an effervescent pair formed from an acid and a carbonate or bicarbonate salt to facilitate dispersion in the liquid medium.

The dosage of the active ingredient of the composition will, of course, vary with the nature of the severity of the condition to be treated and with the particular compound in the composition and its route of administration. It will also vary according to the age, weight and response of the individual patient.

In the embodiments where the pharmaceutical composition comprises trisodium [3-((1S,3R)-1-biphenyl-4-ylmethyl-3-ethoxycarbonyl-1-butylcarbamoyl) propionate-(S)-3′-methyl-2′-(pentanoyl{2″-(tetrazol-5-ylate)biphenyl-4′-ylmethyl}amino)butyrate] hemipentahydrate (LCZ696) in the pharmaceutical compositions for use in the context of the present invention, the unit dose of the therapeutic agents N-(3-carboxy-1-oxopropyl)-(4S)-p-phenylphenylmethyl)-4-amino-(2R)-methylbutanoic acid ethyl ester and valsartan together will be in the range from about 1 to about 1000 mg, such as 40 mg to 400 mg (e.g., 50 mg, 100 mg, 200 mg, 400 mg) per day. Alternatively lower doses may be given, for example doses of 0.5 to 100 mg; 0.5 to 50 mg; or 0.5 to 20 mg per day. (As explanatory note, a unit dose of 100 mg LCZ696 delivering 100 mg of the two agents N-(3-carboxy-1-oxopropyl)-(4S)-p-phenylphenylmethyl)-4-amino-(2R)-methylbutanoic acid ethyl ester and valsartan corresponds to 107.8 mg of trisodium [3-((1S,3R)-1-biphenyl-4-ylmethyl-3-ethoxycarbonyl-1-butylcarbamoyl) propionate-(S)-3′-methyl-Z-(pentanoyl{2″-(tetrazol-5-ylate)biphenyl-4′-ylmethyl}amino)butyrate]hemipentahydrate. Correspondingly, a unit dose of 200 mg requires 215.6 mg, and a unit dose of 400 mg requires 431.2 mg of trisodium [3-((1S,3R)-1-biphenyl-4-ylmethyl-3-ethoxycarbonyl-1-butylcarbamoyl) propionate-(S)-3′-methyl-Z-(pentanoyl{2″-(tetrazol-5-ylate)biphenyl-4′-ylmethyl}amino)butyrate]hemipentahydrate.

Dosages of the sum of the individual compounds (i)/(ii) in the combination of the pharmaceutical composition will be in the range from about 1 to about 1000 mg, such as 40 mg to 400 mg and include but are not limited to 5 mg, 20 mg, 25 mg, 40 mg, 50 mg, 80 mg, 100 mg, 200 mg, 400 mg, 800 mg and 1000 mg. Such dosages for compounds (i)/(ii) can be considered therapeutically effective amounts or dosage strengths. Ratios for the amount of each compound in the pharmaceutical composition are preferably in the about 1:1 molar ratio to achieve an optimal renal protection while still providing cardiovascular benefits.

Pharmaceutical compositions containing a compound of formula(I) (such as compound LCZ696), or compounds (i)/(ii) can be administered any number of times per day, i.e. once a day (q.d.), twice (b.i.d.), three times, four time, etc. in an immediate release formation or less frequently as an extended or sustained release formation. Preferably the pharmaceutical composition is administered twice daily (b.i.d.). Corresponding doses may be taken, for example, in the morning, at mid-day or in the evening.

All the aforementioned embodiments for the methods of protection and treatment according to the present invention are equally applicable to the pharmaceutical compositions for the use in renal protection according to the present invention, to the use of the pharmaceutical compositions for renal protection according to the present invention and to the use of the pharmaceutical compositions for the manufacture of a medicament for renal protection according to the present invention.

The following example is illustrative, but does not serve to limit the scope of the invention described herein.

### Example 1

A 36-week, randomized, double-blind, multi-center, parallel group, active controlled study to evaluate the efficacy, safety, and tolerability of LCZ696 compared to valsartan in patients with chronic heart failure with preserved left-ventricular ejection fraction (HF-PEF).

### LCZ696:

LCZ696 refers to the supramolecular complex trisodium [3-((1S,3R)-1-biphenyl-4-ylmethyl-3-ethoxycarbonyl-1-butylcarbamoyl) propionate-(S)-3′-methyl-2′-(pentanoyl{2″-(tetrazol-5-ylate)biphenyl-4′-ylmethyl}amino)butyrate]hemipentahydrate. This compound and pharmaceutical compositions thereof have been previously disclosed in WO2007/056546 and WO 2009/061713, whose preparative teachings are incorporated herein by reference.

LCZ696 is a first-in-class angiotensin receptor neprilysin inhibitor that comprises the molecular moieties of the NEP (neutral endopeptidase EC 3.4.24.11) inhibitor pro-drug AHU377 (N-(3-carboxy-1-oxopropyl)-(4S)-p-phenylphenylmethyl)-4-amino-(2R)-methylbutanoic acid ethyl ester) and the angiotensin receptor blocker valsartan as a single compound. AHU377 is metabolized by enzymatic cleavage to LBQ657 (N-(3-carboxy-1-oxopropyl)-(4S)-p-phenylphenylmethyl)-4-amino-(2R)-methylbutanoic acid), the active inhibitor of neutral endopeptidase, which is the major enzyme responsible for the breakdown of atrial natriuretic peptides.

### Valsartan:

Valsartan or (S)—N-valeryl-N-{[2′-(1H-tetrazole-5-yl)-biphenyl-4-yl]-methyl}-valine) can be purchased from commercial sources or can be prepared according to known methods, such as described in U.S. Pat. No. 5,399,578 and EP 0443983, whose preparative teachings are incorporated by reference herein.

### Study Design:

Men and women aged 40 years or older with a left ventricular ejection fraction of 45% or above, and a documented history of heart failure with associated signs or symptoms (dyspnea on exertion, orthopnea, paroxysmal dyspnea, and peripheral edema) are eligible. Patients are required to have an NT-proBNP >400 pg/mL at screening, be on diuretic therapy, and have a systolic blood pressure less than 140 mm Hg, or 160 mm Hg or less if on three or more blood pressure medications at randomization. Additional inclusion criteria include an estimated glomerular filtration rate (eGFR) of at least 30 ml/min/1.73 m² at screening (calculated by the Modification of Diet in Renal Disease formula) and a potassium concentration of no more than 5.2 mmol/L.

Patients are excluded if they had prior left ventricular ejection fraction less than 45% at any time, isolated right heart failure due to pulmonary disease, dyspnea due to non-cardiac causes such as pulmonary disease, anemia, or severe obesity, primary valvular or myocardial diseases, or coronary artery or cerebrovascular disease requiring revascularization within 3 months of screening or likely to require revascularization during the trial.

Eligible patients are enrolled into a 2-week, single-blind placebo run-in period, during which time they continue their background medications. ACE inhibitors and angiotensin receptor blockers are required to be discontinued 24 hours prior to randomization. After two weeks, all patients who fulfill the inclusion/exclusion criteria are randomized to either LCZ696 or valsartan in a 1:1 ratio.

Patients are started on LCZ696 50 mg twice daily or valsartan 40 mg twice daily and are titrated to their final medication doses of LCZ696 200 mg twice daily or valsartan 160 mg twice daily over a period of 2 to 4 weeks. Patients are on their starting dose for 1 week and titrated up to either LCZ696 100 mg twice daily or valsartan 80 mg twice daily for 1 week. The maximum LCZ696 dose achieves exposures similar to a dose of valsartan that provides comparable AT1 blockade. At the investigator's discretion, patients are allowed to stay on each titration dose for an additional week. All patients are then titrated to their final doses of LCZ696 200 mg twice daily or valsartan 160 mg twice daily, in addition to standard background therapy. Patients remain on these doses for the remainder of the study, although those not tolerating the maximum dose of study medication could be down-titrated to a lower dose at the Investigator's discretion and then re-challenged to the maximum dose of study medication, or remain on the lower dose.

The dose selection reflects equal exposure of valsartan in both study arms as published by Gu et al., 2010, J Clin Pharmacol., 401-14 and Ruilope et al., 2010, Lancet, 375(9722):1255-66.

### Study Procedures:

The primary study endpoint is the change from baseline in NT-proBNP assessed at 12 weeks, and is analyzed using the last observation after baseline carried forward. Secondary endpoints include changes in echocardiographic measures (left ventricular volumes and ejection fraction, left atrial volume, measures of diastolic function), change in blood pressure, as well as change in New York Heart Association Class (NYHA) clinical composite assessment and quality of life (Kansas City Cardiomyopathy Questionnaire).

Echocardiographic studies are performed at screening, randomization, at week 12, and week 36 or at end of study or early termination visits. Echocardiograms performed at screening are evaluated by local readers for qualifying information. All other echocardiograms are only performed in patients meeting NT-proBNP entry criterion and are evaluated centrally. For assessment of global ventricular size and function, left ventricular end-diastolic and end-systolic volumes are obtained utilizing the Simpson's rule method and left ventricular ejection fraction is derived in the usual fashion. Maximal left atrial dimension is measured in the parasternal long-axis view, and left atrial volume is assessed with the Simpson's rule method and indexed to body surface area. Measurements are made in triplicate in accordance with the recommendations of the American Society of Echocardiography. Blood pressure and heart rate are measured at all study visits with a calibrated standard sphygmomanometer and appropriate size cuff. The use of concomitant medication is recorded at each study visit.

The clinical composite assessment is based on a composite of the NYHA functional classification, patient global assessment and major adverse clinical events. Patients are classified as improved if at the endpoint visit they experienced improvement in NYHA functional classification or in patient global assessment (or both) but do not have a major adverse cardiovascular event. Patients are determined to be worse if at the endpoint visit they experienced a major adverse cardiac event during the double blind treatment or reported worsening of their NYHA class or patient global assessment. Patients are considered unchanged if they are neither improved nor worsened.

### Results:

LCZ696 was well tolerated with adverse effects similar to valsartan.

Of 301 patients, 261 patients completed evaluation at 12 weeks and 241 patients at 36 weeks. The mean age was 71 years, 57% of patients were female, and the majority of patients were NYHA class II. Atrial fibrillation was present at baseline in 85 (28%) patients. Mean left ventricular ejection fraction (LVEF) was 58±7.7%, and LVEF was greater than 50% in 238 (87%) of patients. Blood pressure was well-controlled (mean sitting blood pressure 135/77 mm Hg, median sitting blood pressure 136/79 mm Hg). Baseline NT-proBNP was elevated (geometric mean 830.6 pg/mL, 95% CI 744-928). All patients were on diuretics at baseline and the majority of patients had been taking an ACE inhibitor or angiotensin receptor blocker prior to enrollment. Echocardiographic assessment at baseline demonstrated reduced mitral annular relaxation velocity, elevated E/e′, and enlarged left atria, consistent with mild elevation of cardiac filling pressures.

The primary endpoint, change in NT-proBNP from baseline to 12 weeks, was significantly different in the LCZ696 group compared with the valsartan group (ratio of change LCZ696/valsartan 0.77, 95% CI 0.64 0.92, p=0.005; see Table 1) with a greater reduction in the LCZ696-treated patients.

Analysis of the primary endpoint in completers only (p=0.007) or with multiple imputation for missing values (p=0.01) yielded similar results. The effect of LCZ696 on NT-proBNP occurred fairly early, although an early reduction in NT-proBNP after 4 weeks of treatment in the LCZ696 group compared with the valsartan group was not significant (p=0.063).

The reduction in NT-proBNP at 12 weeks was noted in all prespecified subgroups. Of these subgroups, only patients with diabetes had a differentially greater reduction in NT-proBNP when treated with LCZ696 compared with patients without diabetes (interaction p=0.02).

TABLE 1
NT-proBNP at baseline, 12 weeks, and 36 weeks, and ratio of change in NT-
proBNP at 12 and 36 weeks

| | NT-proBNP (pg/ml) at 12 weeks | | | NT-proBNP (pg/ml) at 36 weeks | | |
|---|---|---|---|---|---|---|
| | n | Baseline | 12 weeks | n | Baseline | 36 weeks |
| LCZ696 | 134 | 783 | 605 | 115 | 763 | 496 |
| | | (670-914) | (512-714) | | (646-901) | (401-613) |
| Valsartan | 132 | 862 | 835 | 116 | 822 | 607 |
| | | (733-1012) | (710-981) | | (688-983) | (484-760) |
| Ratio of change | | | 0.77 (95% CI | | | 0.85 (95% CI |
| (LZC696/valsartan) | | | 0.64-0.92), | | | 0.65-1.09), |
| | | | p = 0.005 | | | p = 0.20 |

Data for NT-proBNP are geometric mean (95% CI)

After 12 weeks of treatment, blood pressure was reduced by 9.3 (SD 14)/4.9 (10) mm Hg in the LCZ696 group and 2.9 (17)/2.1 (11) mm Hg in the valsartan group (p=0.001 for systolic and p=0.09 for diastolic blood pressure differences). LCZ696 was associated with a greater reduction in NT-proBNP than was valsartan even after adjustment for the change in blood pressure between the two groups (p=0.01). Moreover, change in blood pressure correlated poorly with change in NT-proBNP (r=0.104, p=0.1).

Minimal changes in echocardiographic parameters such as left ventricular size or function, diastolic function, left ventricular (LV) mass or tricuspid regurgitant velocity from baseline to 12 weeks between treatment groups has been observed. Left atrial dimension was numerically, but not significantly, reduced at 12 weeks.

Although NT-proBNP remained reduced from baseline at 36 weeks in the LCZ696 group (see Table 1), the difference between treatment groups at 36 weeks was no longer significant (p=0.20; Table 1). At 36 weeks, blood pressure was reduced by 7.5 (15)/5.1 (10.8) in the LCZ696 group versus 1.5 (16)/0.34 (11.5) in the valsartan group (p=0.006 for systolic and p=0.001 for diastolic blood pressure differences).

Left atrial volume and left atrial volume index (LAVI) was reduced significantly in the LCZ696 group after 36 weeks of treatment (p=0.003 and 0.007, for left atrial volume and LAVI respectively), as was left atrial dimension (p=0.034) (Table 2).

The change in left atrial size was most apparent in patients without atrial fibrillation at baseline. No other echocardiographic measures, including LVEF, ventricular volumes, left ventricular mass index, relative wall thickness, or measures of diastolic function, differed between treatment groups at 36 weeks.

TABLE 2
Changes in Echocardiographic Parameters at 36 weeks (Left atrial
dimension (LA dimension), left atrial volume (LA Volume), left atrial
volume index (LA Volume Index), left ventricle mass index (LA mass
index) and relative wall thickness)

| | LCZ696 | | | Valsartan | | | |
|---|---|---|---|---|---|---|---|
| | Baseline | | Δ from | Baseline | | Δ from | |
| | N | | Baseline | N | | Baseline | p-value |
| LA | 99 | 3.68 | −0.15 | 108 | 3.73 | −0.08 | 0.03 |
| dimension | | | | | | | |
| (cm) | | | | | | | |
| LA Volume | 96 | 65.26 | −4.61 | 112 | 68.28 | 0.37 | 0.003 |
| (ml) | | | | | | | |
| LA Volume | 90 | 35.01 | −2.61 | 106 | 36.80 | 0.31 | 0.007 |
| Index | | | | | | | |
| (ml/m²) | | | | | | | |
| LV mass | 91 | 76.56 | −2.78 | 100 | 79.45 | −1.93 | 0.35 |
| index | | | | | | | |
| (g/m²) | | | | | | | |
| Relative wall | 98 | 0.37 | +0.01 | 107 | 0.37 | +0.01 | 0.96 |
| thickness | | | | | | | |
| (%) | | | | | | | |

NYHA class improvement at 12 weeks did not differ significantly between groups (p=0.11), but we noted an improvement in NYHA class at 36 weeks in the LCZ696 group compared with the valsartan group (p=0.05). Clinical composite assessment after 12 weeks (p=0.19) and 36 weeks (p=0.17) of treatment did not differ significantly between groups. There was no difference in KCCQ score between treatment groups at either time point.

Target dose was achieved in 121 (81%) patients in the LCZ696 group and in 119 (78%) in the valsartan group. The use of concomitant blood-pressure lowering drugs, particularly loop diuretics, was greater in the valsartan group during the trial, although β-blocker use was similar. In the LCZ696 group, 22 patients (15%) had one or more serious adverse events, including one death; in the valsartan group, 30 patients (20%) had one or more serious adverse events, including two deaths. In the valsartan group, the adverse event “atrial fibrillation” was observed in 8 patients (5.3%), whereas in the LCZ696 group, only 2 patients (2.0%) experienced this adverse event.

The number of patients with hypotension, renal dysfunction, or hyperkalaemia did not differ between groups.

Over 36 weeks, eGFR decreased to a greater extent in the valsartan group (LCZ696, −1.6 mL/min per 1.73 m² vs valsartan, −5.2 mL/min per 1.73 m²; p=0.007) and serum creatinine also increased to a greater extend in the valsartan group (Table 3), whereas urinary albumin creatinine ratio increased to a greater extent in the LCZ696 group (LCZ696, 1.9 mg/mmol at baseline, 2.9 mg/mmol at week 36; valsartan, 2.0 mg/mmol at baseline, 2.0 mg/mmol at week 36; p=0.02).

TABLE 3
Changes in renal function parameters at 36 weeks (eGFR and serum creatinine)

| | LCZ696 | | Valsartan | | LCZ696 vs. Valsartan | |
|---|---|---|---|---|---|---|
| | N = 127 | | N = 125 | | LSM of | |
| | | LSM of CFB | | LSM of CFB | difference | |
| | N | (SE) | N | (SE) | (95% CI) | P-value |
| eGFR | 126 | −3.68 (1.493) | 123 | −7.14 (1.517) | 3.46 (0.64, 6.27) | 0.0163* |
| (mL/min/1.73 m²) | | | | | | |
| Serum creatinine | 127 | 5.82 (2.136) | 125 | 10.65 (2.183) | −4.83 (−8.87, −0.79) | 0.0193* |
| (μmol/L) | | | | | | |

Change from baseline (CFB) = Endpoint − Baseline;
LSM = Least squares mean;
CI = Confidence interval;
SE = Standard error.
*Indicates statistical significance at 0.05 level.

Angio-oedema occurred in one patient on LCZ696, who did not need admission to hospital, and no patients on valsartan.

### CONCLUSION

In summary, in patients with heart failure with preserved ejection fraction, the angiotensin receptor neprilysin inhibitor LCZ696 reduced NT-proBNP to a greater extent than valsartan after 12 weeks of therapy. The reduction in NT-proBNP in patients receiving LCZ696 became evident at 4 weeks and was sustained to 36 weeks, though the between group difference was no longer statistically significant. We further observed a reduction in left atrial size, indicative of reverse left atrial remodeling, in patients randomized to LCZ696 after 36 weeks, compared with those randomized to valsartan. We observed trends in improvement in NYHA class in those patients randomized to LCZ696, which was overall well-tolerated.

In addition, in patients with HFpEF, the angiotensin receptor neprilysin inhibitor LCZ696 resulted in a larger drop in SBP and DBP compared with valsartan alone. Furthermore, LCZ696 better preserved renal function compared with valsartan after 36 weeks of therapy as shown with the eGFR and the creatinine data. The preservation of renal function in patients receiving LCZ696 indicates that its administration may have favorable effects in patients with HF-pEF.

### Example 2

A randomized, double-blind, parallel group, active-controlled, two-arm, event-driven trial comparing the long-term efficacy and safety of enalapril and LCZ696 on morbidity and mortality in patients with chronic symptomatic heart failure and reduced ejection fraction (HF-REF) [PARADIGM-HF].

### LCZ696: see Example 1

### Enalapril

The ACE inhibitor Enalapril or (2S)-1-[(2S)-2-{[(2S)-1-ethoxy-1-oxo-4-phenylbutan-2-yl]amino}propanoyl]-pyrrolidine-2-carboxylic acid can be purchased from commercial sources or can be prepared according to known methods.

### Obiective & Methods:

Patients with chronic HF, NYHA functional class II-IV symptoms, an elevated plasma B-type natriuretic peptide (BNP) or NT-proBNP level and, initially, a left ventricular ejection fraction of 40% (later amended to 35%) are eligible. Patients enter a single blind enalapril run-in period (titrated to 10 mg bid) which, depending on tolerability, is followed by an LCZ696 run-in period (100 mg titrated to 200 mg bid). Then, patients tolerating both drugs at the target dose, are randomized 1:1 to either enalapril 10 mg bid or LCZ696 200 mg bid. The primary outcome is the composite of cardiovascular death or HF hospitalization, although the trial is powered to detect a 15% relative risk reduction in cardiovascular death with LCZ696, compared with enalapril. Secondary outcome measures are change in the Kansas City Cardiomyopathy Questionnaire (KCCQ) clinical summary score at 8 months, change in renal function, and time to all-cause mortality.

### Study Design and Procedures

Detailed study design and procedures can be found under www.clinicaltrials.gov, study number NCT01035255, and as published in The European Journal of Heart Failure by McMurray et al (18 Apr. 2013) titled “Dual angiotensin receptor and neprilysin inhibition as an alternative to angiotensin converting enzyme inhibition in patients with chronic systolic heart failure: rationale for and design of the Prospective comparison of ARNI with ACEI to Determine Impact on Global Mortality and morbidity in Heart Failure trial (PARADIGM-HF)”.

### Example 3

A multicenter, randomized, double-blind, parallel group, active-controlled study to evaluate the efficacy and safety of LCZ696 compared to valsartan, on morbidity and mortality in heart failure patients (NYHA Class 11-1V) with preserved ejection fraction [PARAGON-HF]

### LCZ696: See Example 1

### Valsartan: See Example 1

### Background

Heart failure with preserved ejection fraction (HFpEF) accounts for up to half of heart failure (HF) cases and is associated with substantial morbidity and mortality. To date both angiotensin converting enzyme inhibitors (ACEIs) and angiotensin receptor blockers (ARBs) have been tested in clinical trials in HFpEF and not been shown to improve the primary outcome. Several pathophysiologic mechanisms have been implicated in this disorder, including abnormalities of diastolic function and impaired natriuretic response to acute volume expansion.

LCZ696 is a first in class, angiotensin receptor neprilysin inhibitor (ARNI), providing systemic exposure to AHU377, a neprilysin (NEP) inhibitor and valsartan, an ARB. The potential clinical benefits from NEP inhibition can only be leveraged if the RAAS system is inhibited concomitantly.1, 2

The mechanisms of action of LCZ696 suggest that it may have an impact on the pathophysiology of HFpEF, in which it is believed that excessive fibrosis and myocyte hypertrophy lead to abnormal left ventricular relaxation and filling, impaired diastolic distensibility and/or increased vascular stiffness, with consequent elevated cardiac filling pressures.

The PARAMOUNT trial tested the safety and efficacy of LCZ696 in patients with HFpEF and showed a significant reduction in N-terminal pro-B-type natriuretic peptide (NT-proBNP) at 12 weeks and significant improvement in left atrial size and New York Heart Association (NYHA) class in patients randomized to LCZ696 compared to valsartan at 36 weeks. NT-proBNP is not a substrate for neprilysin.

### Methods

PARAGON-HF will assess the effect of LCZ696 on outcomes (cardiovascular [CV] death and total first and recurrent HF hospitalizations) in patients with HFpEF.
Screening: up to 2 weeks
Active Run-In Period: 3-8 weeks (can be shorter for patients previously exposed to standard doses of RAAS blockade; longer for patients with no prior exposure or on low doses of ACEIs or ARBs.)
Double Blind Period: Projected 2.75 years enrollment; with a minimum of 2 years follow up

### Primary and Secondary Obiectives

Primary objective: The primary objective of this trial is to compare LCZ696 to valsartan in reducing the rate of the composite endpoint of CV death and total (first and recurrent) HF hospitalizations, in HF patients (NYHA Class II-IV) with preserved EF (left ventricular ejection fraction [LVEF]≧45%).

### Secondary Objectives:

To compare LCZ696 to valsartan in reducing the rate of the composite endpoint of CV death, total HF hospitalizations, total non-fatal strokes, and total non-fatal myocardial infarctions (MIs). Total is defined as the first and all recurrent events.
To compare LCZ696 to valsartan in improving NYHA functional classification at 8 months.
To compare LCZ696 to valsartan in delaying the time to new onset AF in patients with no history of AF and without AF on electrocardiogram (ECG) at baseline.
To compare LCZ696 to valsartan in delaying the time to all-cause mortality.

### Study Design and Detailed Procedures

Detailed study design and procedures can be found under www.clinicaltrials.gov, study number NCT01920711.

1. A method for the protection of the kidney of a mammal suffering from hypertension or heart failure or being prone to suffering from hypertension and/or heart failure comprising administering to said mammal in need of such protection a pharmaceutical composition comprising a therapeutically effective amount of
a) the compound trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate (LCZ696),
or
b) a combination comprising a physical mixture of
(i) valsartan or a pharmaceutically acceptable salt thereof; and
(ii) N-(3-carboxy-1-oxopropyl)-(4S)-(p-phenylphenylmethyl)-4-amino-2R-methylbutanoic acid ethyl ester or a pharmaceutically acceptable salt thereof,
in a 1:1 molar ratio.

2. The method according to claim 1 wherein the mammal is a human.

3. The method according to claim 1, wherein the heart failure is congestive heart failure, left heart failure, right heart failure, chronic heart failure, advanced heart failure, acute heart failure, acute decompensated heart failure, heart failure with reduced ejection fraction, or heart failure with preserved ejection fraction.

4. The method according to claim 3, wherein the heart failure is heart failure with reduced ejection fraction (HF-rEF).

5. The method according to claim 3, wherein the heart failure is heart failure with preserved ejection fraction (HF-pEF).

6. The method according to claim 1, wherein the mammal suffers from hypertension.

7. The method according to claim 1, wherein the therapeutically effective amount of compound of formula (I) or of the combination is effective to provide renal protection to the mammal.

8. The method according to claim 1, wherein the therapeutically effective amount of compound of formula (I) or of the combination is effective to induce at least one physiological effect in the mammal including vasodilation, diuresis, natriuesis and combinations thereof.

9. The method according to claim 1, wherein the administration of a therapeutically effective amount of compound of formula (I) or of the combination better preserves renal function than administration of the corresponding amount of an angiotensin receptor blocker, such as valsartan, alone.

10. The method according to claim 1, wherein the therapeutically effective amount of compound of formula (I) or of the combination slows the time to change in renal function defined by loss of estimated glomerular filtration rate (eGFR) in addition to reducing CV mortality and HF hospitalization in comparison to the administration of the corresponding amount of an angiotensin receptor blocker, such as valsartan, alone.

11. The method according to claim 1, wherein the administration of a therapeutically effective amount of compound of formula (I) or of the combination induces a lesser decrease of the estimated glomerular filtration rate (eGFR) than administration of the corresponding amount of an angiotensin receptor blocker, such as valsartan, alone.

12. The method according to claim 1, wherein the administration of a therapeutically effective amount of compound of formula (I) or of the combination slows down the decrease of the estimated glomerular filtration rate (eGFR) in patients suffering from heart failure.

13. The method according to claim 1, wherein the administration of a therapeutically effective amount of compound of formula (I) or of the combination induces a slower decrease of the estimated glomerular filtration rate (eGFR) in patients suffering from heart failure than administration of the corresponding amount of an angiotensin receptor blocker, such as valsartan, alone.

14. The method according to claim 1, wherein the administration of a therapeutically effective amount of compound of formula (I) or of the combination is effective to achieve a reduction of the left atrial volume, the left atrial volume index (LAVI) and/or of the left atrial dimension which is larger than upon administration of the corresponding amount of an angiotensin receptor blocker, such as valsartan, alone.

15. The method according to claim 1, wherein the pharmaceutical composition comprises in addition one or more pharmaceutically acceptable carriers.

16. The method according to claim 1, wherein the pharmaceutical composition comprises the compound trisodium [3-((1S,3R)-1-biphenyl-4-ylmethyl-3-ethoxycarbonyl-1-butylcarbamoyl)propionate-(S)-3′-methyl-2′-(Pentanoyl{2″-(tetrazol-5-ylate)biphenyl-4′-ylmethyl}amino)butyrate] hemipentahydrate (LCZ696).

17. The method according to claim 16, wherein the pharmaceutical composition is administered to deliver a daily overall dose of LCZ696 from 50 mg to 400 mg.

18. The method according to claim 16, wherein the pharmaceutical composition is administered to deliver LCZ696 twice daily with a dose of 50 mg, 100 mg, or 200 mg.

19.-26. (canceled)
